(19) United States
(12) Patent Application Publication
(10) Pub. No.: US 2004/0120900 A1
(43) Pub. Date: Jun. 24, 2004

(54) Intraoral calculus dissolving gel composition

(76) Inventor: Peter Arsenault, Dracut, MA (US)

Correspondence Address: Joseph E. Funk, PO Box 661, Londonderry, NH, 03053, US

(21) Appl. No.: 10/324,668

(22) Filed: Dec. 20, 2002

(51) Int. Cl. A61K 7/28

(52) U.S. Cl. 424/50

(57) ABSTRACT

A dental composition in gel form is taught that is applied to a user's teeth for a period of time using a dental tray. The gel utilizes the enzyme papaine to soften calculus on the teeth so that it may be removed by tooth brushing, flossing and rinsing.

### FIELD OF THE INVENTION

This invention relates to oral hygiene, and more particularly to a gel for softening of calculus/tartar on teeth for easy removal during normal tooth brushing, flossing and rinsing.

### BACKGROUND OF THE INVENTION

At this time dental disease is the most common human disease worldwide as recognized by the World Health Organization. A large proportion of the population of many countries, and the population of some countries more than others, have tooth decay (caries) and gum (periodontal) disease.

Periodontal diseases are the most prevalent chronic diseases affecting individuals of all ages from children to the elderly. It has been estimated that one-half of all adults in the U.S. have inflammation of the gums (gingivitis), the first stage of periodontal disease. Eighty percent of all adults have had some degree of gum inflammation that has led to the destruction of the bone which supports the teeth, which, as if it progresses can lead to tooth loss.

A contributing factor to both tooth decay and periodontal disease is dental plaque. Dental plaque comprises bacteria within a matrix of food particles and other organic materials, and builds up on the surfaces of teeth as a film of a spongy mass with an uneven surface. Prevention of dental disease involves oral hygiene practices to remove plaque and prevent calculus/tartar. Plaque that remains on a person's teeth calcifies to form calculus/tartar. It is important to remove the plaque before it calcifies into calculus because the removal of calculus requires more extensive effort and care for its removal, usually by a dental health professional. Removal of calculus is done by scraping with dental instruments or the use of sonic or ultrasonic instruments.

Personal oral hygiene practices such as proper tooth brushing and flossing are the best ways to remove plaque and thereby minimize the development of calculus. Due to the difficulty many people have in adopting and maintaining good oral hygiene measures they too often fail to remove plaque adequately. In addition, too many people do not perform all these good oral hygiene practices, ever. Consequently, calculus accumulates and the above-described dental problems occur.

Accordingly, there is a need in the art for a product that can aid in the removal of calculus by making it easy to remove by tooth brushing, flossing and rinsing instead of the existing techniques.

### SUMMARY OF THE INVENTION

The present invention is a dental gel composition that is applied to calculus and softens it, thus making it removable by tooth brushing, flossing and risning. The novel composition is applied via a dental tray, such as a bleaching tray, to a person's teeth for a period of time, as prescribed by a dentist, to soften the calculus on the person's teeth for removal by tooth brushing, flossing and rinsing. The active ingredient for softening calculus is the enzyme papaine.

### DETAILED DESCRIPTION

The base or carrier ingredient in the calculus (calcified plaque) softening dental gel composition is a carbomer, such as one of the 900 series of carbomers available from B. F. Goodrich under the trademark Carbopol. Carbopol is a carboxyvinyl polymer formed of a water soluble vinyl polymer gel base. More particularly, Carbopol is a polymer of acrylic acid cross linked with allylsucrose, bentonite, soluble cellulose derivatives (e.g. sodium carboxy methyl cellulose, hydroxypropyl methyl cellulose), “Veegum” or polyvinyl alcohol.

To produce a gel at least some of the carboxyl groups of the Carbopol should be neutralised and we prefer to use an organic base, e.g. triethylamine or diisopropanolamine, which forms a salt with the Carbopol.

Carbopol is often used as a thickening agent in dental compositions but it also has a lesser known property as an absorbefacient or surface active agent. Other materials that have this absorbefacient property are sodium lauryl sulfate, sodium dodecylbenzene sulfonate, sodium alkyldiphenyl ether difulfonate, dioctyl sodium sulfosuccinate, and polyoxyalkylphenyl ether sulfate ammonium salt; such alcohols as ethanol, glycerin, diethylene glycol, propylene glycol, polyethylene glycol, and higher fatty acid alcohol; dimethyl sulfoxide and alkylmethyl derivatives; salicylic acid, urea, dimethylacetamide, diethyl toluamide, dimethylformamide, dioctyl sebacate, lanolin, allantoin, squalene, diisopropyl adipate, pyroglutamic acid lauryl ester, ethyl laurate, methyl nicotinate, sorbitol, pyrrolidone derivatives like dodecyl pyrrolidone and methyl pyrrolidone, olive oil, castor oil, liquid paraffin, vaseline, gelatin, amino acid, and benzyl nicotinate. As an absorbefacient Carbopol is an absorption promotor or diffusion promoter.

Carbopol is added to the novel dental composition in the amount of 1.5 grams to make up 100 milliliters of the novel calculus softening gel.

The enzyme Papaine is the active ingredient that is added to the novel dental composition to aid in softening calculus for removal by tooth brushing, flossing and rinsing.

Enzymes are biological catalysts of chemical reactions in living systems. Enzymes combine with the substrates on which they act forming an intermediate enzyme-substrate complex. This complex is then converted to a reaction product and a liberated enzyme which continues its specific enzymatic function.

Enzymes provide several benefits when used for cleansing of the oral cavity. They break down salivary proteins which are adsorbed onto the tooth surface and form the pellicle; the first layer of resulting plaque. Enzymes along with lipases destroy bacteria by lysing proteins and lipids which form the structural component of bacterial cell walls and membranes. Dextranases break down the organic skeletal structure produced by bacteria that forms a matrix for bacterial adhesion. Proteases and amylases, not only prevent plaque formation, but also prevent the development of calculus by breaking-up the carbohydrate-protein complex that binds calcium, preventing mineralization.

Although Papaine is the preferred enzyme for the novel dental gel composition, other enzymes may be used. Useful enzymes include any of the commercially available proteases, dextranases, glucanohydrolases, endoglycosidases, amylases, mutanases, lipases and mucinases or compatible mixtures thereof. One gram of the preferred enzyme, papaine, or a mixture of several compatible enzymes, is added to make up each 100 milliliters of the novel calculus softening gel.

The novel dental composition also contains a sweetening agent or flavorant. Examples of sweetening agents are levaudioside, neohesperidyl dihydrochalcone, acesulfam, taumatine, glycyrrhizin, glycyrrhizin monogluconide, hernandulcin, perillartine, saccharin, saccharin sodium, dextrose, sorbitol, Aspartame, xylitol, stevioside, stevia extract, Acesulfame, starch syrup, alone or in admixture. The preferred sweetening agents are a naturally occurring terpene glycoside which can be extracted from members of the cucurbitaceae family, preferably from Siraitia grosvenori which produces a kiwi-like fruit, and diterpene glycosides from the rubus family, preferably from the genus/species rubus suavissimus, blackberry, kiwi, raspberry, blueberry and other genus/species of the rubus family. The preferred sweetening agent in this novel dental composition is stevia extract obtained from stevia rebaudiana. Stevia extract is used in the inventive formulation for its natural sweetness, while at the same time inhibiting the formation of plaque on teeth. These sweetening agents are illustrative only and not meant to imply they are the most suitable or the only products that may be used. The amount of sweetener used in making the novel dental composition depends on how sweet it is desired to make the novel dental gel composition.

Depending on the desired thickness of the calculus softening gel, thickeners may be added to the composition to increase the thickness of the Carbopol. Thickeners that may be added include Hyroxyethyl cellulose, alginic acid salts and esters, locust bean gum, guar gum, gum tragacynth, carboxymethyl cellulose, polyvinyl acetate, polyvinyl pyrrolidone, aerogels of alumina and silica, and the like. These thickeners are illustrative only and not meant to imply they are the most suitable or the only products that may be used. The amount that is uses, if any, depends on if it is desired to make the dental gel composition thicker than the carbopol makes it.

The novel dental composition may also have its pH adjusted. The preferred pH is 6.3 but the invention is not dependent on an exact pH of the gel composition. The pH adjusting agents that may be used include, but are not limited to, organic acids and salts thereof such as citric acid and its salts, phosphoric acid and its salts, malic acid and its salts, and acetic acid and its salts, gluconic acid and salts thereof, maleic acid and salts thereof, aspartic acid and salts thereof, gluconic acid and salts thereof, succinic acid and salts thereof, glucuronic acid and salts thereof, fumaric acid and salts thereof, glutamic acid and salts thereof, adipic acid and salts thereof, lactic acid and salts thereof, and pantothenic acid and salts thereof, alone or in admixture. These pH adjusting agents are illustrative only and not meant to imply they are the most suitable or the only products that may be used.

A stabilizing agent is also added to the dental composition. This agent maintains the dental composition in its emulsified state. The preferred stabilizing/emulsifying agent is trolamine of which 0.6 milliliters is added to make up the 100 milliliters of the novel dental gel composition. Other such agents that may be utilized are, for example, acacia, diethanolamine, glyceryl monostearate, lanolin alcohols, lecithin, mono- and di-glycerides, mono-ethanolamine, oleic acid, oleyl alcohol, poloxamer, Pluronics.RTM. (BASF, Parsippany, N.J.), polyoxyethylene 50 stearate, polyoxyl 35, castor oil, polyoxl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate, stearic acid, and emulsifying wax. These stabilizing agents are illustrative only and not meant to imply they are the most suitable or the only products that may be used.

Other optional ingredients that can be used without deleteriously affecting, and in some cases even enhancing, the efficacy of the formulations of the novel dental composition include, but are not limited to, acidifying agents such as acetic acid, citric acid, fumaric acid, lactic acid and nitric acid; alkalinizing agents such as ammonia solution, ammonium carbonate, diethanolamine, monoethanolamine, potassium hydroxide, sodium borate, sodium carbonate, sodium hydroxide, and triethanolamine; buffering agents such as potassium metaphosphate, potassium phosphate, sodium acetate and sodium citrate; antioxidants such as ascorbic acid, ascorbyl palmitate, butylated hydroxyanisole, butylated hydroxytoluene, hypophosphorous acid, monothioglyceride, propyl gallate, sodium ascorbate, sodium bisulfite, sodium formaldehyde sulfoxylate and sodium metabisulfite; chelating agents such as edetate disodium and edetic acid; colorants such as FD&C Red No. 3, FD&C Red No. 20, FD&C Yellow No. 6, FD&C Blue No. 2, D&C Green No. 5, D&C Orange No. 5, caramel and ferric oxide red; and flavoring agents such as anise oil, cinnamon oil, cocoa, menthol, orange oil, peppermint oil vanillin. Suitable concentrations for use will be apparent to those of skill in the art. Other optional ingredients, as well as suitable concentrations for use, can be found, for example, in Remington's Pharmaceutical Sciences, latest edition, Mack Publishing Co., Easton, Pa.

To apply the novel dental composition gel to a person's teeth to loosen calculus adhered thereto, a dental tray is preferably used. Such dental trays are well known in the art. A custom fit tray is preferred. It is based on an impression of the patients teeth where models of the teeth are made then trays are fabricated. Also, over the counter or similar type trays known in the art may be used, but a custom fit tray is preferred. The period of treatment is determined by a dentist and may be prescribed for several hours during the day, overnight, or however deemed necessary by a dentist.

Depending on the buildup of calculus on a person's teeth, a dentist may prescribe that the dental composition gel of the present invention be applied more frequently for a time, and for longer periods of time, but within safe limits, until the calculus is removed. Thereafter, the dentist will prescribe an appropriate maintenance schedule for the gel application to manage and prevent further buildup of calculus.

An advantage of this novel gel is such that the gel has the ability to flow into open periodontal pockets and in and around periodontal defects where the toothbrush and floss cannot reach. This results in the calculus being brought into solution for rinsing away.

While what has been disclosed herein is the preferred embodiment of the invention, it will be obvious to those skilled in the art that numerous changes may made be made without departing from the spirit and scope of the invention.

What is claimed is:

1. A dental composition providing improved teeth cleansing properties, the dental composition comprising:
a safe and effective amount of a thickening agent;
a safe and effective amount of an enzyme used for dental applications; and
wherein said dental composition is applied to the teeth for a period of time to soften calculus on the teeth so the calculus can be removed by brushing, flossing and rinsing of the teeth.

2. The invention in accordance with claim 1 wherein said dental composition is in gel form and is applied to the teeth using a dental tray.

3. The invention in accordance with claim 2 wherein the dental composition in gel form is applied to the teeth using the dental tray for a number of hours as prescribed by a dentist.

4. The invention in accordance with claim 3 wherein the enzyme is papaine.

5. The invention in accordance with claim 4 wherein the thickening agent is a carboxyvinyl polymer.

6. The invention in accordance with claim 1 wherein the enzyme is selected from the group(s) consisting of endoglycosidase, papain, dextranase, mutanase, proteases, glucanohydrolases, amylases, lipases and mucinases and mixtures thereof.

7. The invention in accordance with claim 6 wherein the thickening agent is selected from the group(s) consisting of Hyroxyethyl cellulose, alginic acid salts and esters, locust bean gum, guar gum, gum tragacynth, carboxymethyl cellulose, polyvinyl acetate, polyvinyl pyrrolidone, aerogels of alumina and silica and mixtures thereof.

8. The invention in accordance with claim 7 further comprising a stabilizing agent selected from acacia, diethanolamine, glyceryl monostearate, lanolin alcohols, lecithin, mono- and di-glycerides, mono-ethanolamine, oleic acid, oleyl alcohol, poloxamer, Pluronics.RTM. (BASF, Parsippany, N.J.), polyoxyethylene 50 stearate, polyoxyl 35, castor oil, polyoxl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate, stearic acid, emulsifying wax, and mixtures thereof.

9. The invention in accordance with claim 8 further comprising a flavorant selected from levaudioside, neohesperidyl dihydrochalcone, acesulfam, taumatine, glycyrrhizin, glycyrrhizin monogluconide, hernandulcin, perillartine, saccharin, saccharin sodium, dextrose, sorbitol, Aspartame, xylitol, stevioside, stevia extract, Acesulfame, starch syrup, and mixtures thereof.

10. A method for softening calculus on teeth so it can be removed by brushing, flossing and rinsing the teeth comprising the step of:
applying a gel containing a safe and effective amount of an enzyme to the teeth for a number of hours using a dental tray as prescribed by a dentist.

11. The method in accordance with claim 10 wherein the gel is thickened by a thickening agent of carboxyvinyl polymer.

12. The method in accordance with claim 11 wherein the enzyme is papaine.

13. The method in accordance with claim 11 wherein the carboxyvinyl polymer is provided in the amount of 1.5 grams to make up 100 milliliters of the gel with enzyme.

14. The method in accordance with claim 12 wherein a sufficient amount of papaine is added to the carboxyvinyl polymer to make up the 100 milliliters of the gel with enzyme.

15. The method in accordance with claim 12 where 0.6 milliliters of the stabilizing agent trolamine is added to make up the 100 milliliters of the gel with enzyme.
